(19) United States
(12) Patent Application Publication
(10) Pub. No.: US 2009/0248578 A1
(43) Pub. Date: Oct. 1, 2009

(54) METHODS AND APPARATUS FOR MEDICAL DEVICE INVESTMENT RECOVERY

(75) Inventors: Cameron Pollock, Sammamish, WA (US); Tanar Ulric, Woodinville, WA (US); Jonathan Slobodzian, Lynnwood, WA (US)

Correspondence Address: TOWNSEND AND TOWNSEND AND CREW LLP;LIPOSONIX, INC. (70353), TWO EMABARCADERO CENTER, EIGHTH FLOOR, SAN FRANCISCO, CA, 94111, US

(73) Assignee: LipoSonix, Inc., Bothel, WA (US)

(21) Appl. No.: 12/407,212

(22) Filed: Mar. 19, 2009

Related U.S. Application Data
(60) Provisional application No. 61/038,334, filed on Mar. 20, 2008.

(51) Int. Cl. G06Q 40/00; G06Q 20/00; A61B 8/00

(52) U.S. Cl. 705/64; 705/4; 600/437

(57) ABSTRACT

A method of providing a consumer or user with an investment recovery from a pay-per-use device. The investment recovery system involves a computer implemented method of determining the residual number of uses on a product, and crediting a user with the residual number. Also described are apparatus and systems for use with the method of providing medical device investment recovery.

### CROSS-REFERENCES TO RELATED APPLICATIONS

This application claims the benefit of provisional application No. 61/038,334 (Attorney Docket No. 021356-003500US), filed on Mar. 20, 2008, the full disclosure of which is incorporated herein by reference.

### BACKGROUND

Medical products are sometimes sold to physicians, clinics, hospitals or other end users (users) with a pay-per-use (PPU) arrangement. That is, a product or medical apparatus, system or service, is sold to users with a discrete cost assigned to each and every use. For example, a user may purchase a product for a cosmetic system and pay for a definite number of uses. The system to which the product attaches keeps track of the number of uses, and decrements the use life of the product each time the device is used. This is often accomplished using a memory chip on the PPU product. The system used with the product can communicate with the memory chip and decrement the product life according to the system program parameters.

In PPU devices, the user must pay for each allowed use of the device. Instruments and medical devices such as system components designed for multiple uses on a single patient, or components designed for limited use due to breakage or disposability issues, are purchased with a preset limit on the number of uses allowed. A PPU device is disabled when the number of uses is exhausted. Disabling the device can be an inconvenience if the product runs out of life in the middle of a patient procedure. To reduce the inconvenience to the patient, users are known to discard or replace the PPU when it gets “low.” The low value PPU is often disposable, so the device is discarded. The user has paid-for value on the device which is discarded, and that cost is generally passed on to the patient.

Existing PPU devices and systems that use PPU products are able to track the total number of paid uses when the PPU component is new, and track how many have been used, and how many remain. However these systems do not have the ability to decrement the memory chip unless the system is in use for the appropriate medical service. Therefore, it is not possible for these systems to decrement the memory chip or memory means and provide a credit to the user. Examples of various products and methods related to PPU devices are found in U.S. Pat. Nos. 6,446,048; 6,687,679; 6,748,067; 6,910,020; 6,985,879; and U.S. patent application Ser. Nos. 10/045,151; 10/824,935; 10/943,109; 10/943,110; 10/926,779; 10/734,046; 11/053,394 and 11/461,236.

This arrangement contributes to increased health care costs, and produces undesirable effects for individual purchasers of medical treatments, and an aggravated cost on society as a whole.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In accordance with an embodiment, a computer implemented pay-per-use (PPU) investment recovery method is provided for use with medical systems. The method involves receiving a command for a medical system, the command requesting a PPU data storage and clearing operation from a PPU product, the product being a component of the medical system. Once the command is received, the total PPU data value (v) is read from the product, and an investment recovery value (x) being less than or equal to the total PPU data value (v) is stored into a memory device. The investment recovery value (x) is then cleared from the product by subtracting the investment recovery value (x) from the total PPU data value (v) and a credit is provided to the user based on the investment recovery value (x).

The credit may be provided to the user through the medical system, or through a remote location, such as a remote server located at a supplier or other business capable of monitoring and verifying the data accuracy of the investment recovery value (x). If the credit is provided by a remote server, the server has a library of user accounts. The investment recovery data (x) is entered into the appropriate user account, summed with the existing user account value B and the sum checked against a table of possible investment return options. These options include, but are not limited too, credit for parts or services for the medical device, the PPU product/consumable, related products or services, cash or other “rewards” as may be desired.

The computer implemented investment recovery program operates using an apparatus or product having an investment recovery capability. The product has an enclosure adapted for removable engagement with a medical system. An electrical circuit is used within the enclosure, the circuit having at least one port for data communication with the exterior of the enclosure. A component for use in the medical system is suspended within the enclosure, the component requiring an activation signal, and including a read-write (RW) data storage device incorporated into the electrical circuit. The data storage device includes a preset number of uses that can be modified when the component is activated or used.

There is also described a medical system having a product with an investment recovery capability, an electronic controller for operating the product and the component, the electronic controller being able to read and write data to the RW storage device incorporated into the product, and adjunct systems as needed for the operation of the product and component in order for the medical system to perform its intended function.

Where the medical device is purchased in a pay-per-use manner, the use value on the product can be recovered at some value relative to the original cost of the product, and credited to the user. The product has a component integrated into it that is a critical operating component of the medical system. The component may be a critical operating element to the main task of the medical system (e.g., an ultrasound transducer for an ultrasound medical system) or it may be an adjunct critical component for system operation (e.g., a filter for a water purification system or a power regulator for an electrically driven motor). In an embodiment, the component is an element of the system that is removable, and one that the system cannot operate without.

A user may select to recover a portion or all of the use values on the product and receive credit for them. Crediting may occur via a direct exchange of the credit value for an award (or credit) from a credit sponsor, or may involve a credit database having additional information to provide expanded customer service to the customer or user of the medical device.

A method is provided for determining the proportion of value to be refunded or rewarded to a user upon making a claim for a reward. A system incorporating the methods disclosed is also described.

Additional embodiments and variations are herein described.

For a fuller understanding of the nature and advantages of the present invention, reference should be made to the ensuing detailed description and accompanying drawings.

### BRIEF DESCRIPTION OF THE DRAWINGS

FIG. 1 illustrates a purchase and use model of the prior art.

FIG. 2 provides a purchase and use model incorporating the present invention.

FIG. 3 provides a basic view of the method for recovering value from a medical device.

FIG. 4 shows an embodiment of recovery for multiple devices.

FIG. 5 provides an illustration of a crediting method with a tracking system.

FIG. 6 illustrates a method of recovery linked to an award system.

FIG. 7A shows the path of a medical device after the use value has been reduced by a recovery.

FIG. 7B shows a method of increasing a use value of a product.

FIG. 7C shows a web portal and remote server for use with a PPU crediting process.

FIG. 8 shows an embodiment of the interaction with the user to initiate recovery when the medical device is a transducer and the RW storage is within a medical product.

FIG. 9A shows an embodiment of a medical system having a PPU recovery system.

FIG. 9B shows a therapy head incorporating a PPU Product.

FIG. 10 shows an example of a PPU product integrated into a medical device with a recovery system and method.

FIG. 11 shows an example of a PPU product value check.

### DETAILED DESCRIPTION

Described herein are methods and apparatus for the recovery of PPU investment in medical devices. The method associated with recovery of PPU investments utilizes a computer implemented pay-per-use (PPU) investment recovery method for use with medical systems. The method receives a command for a medical system, the command requesting a PPU data storage and clearing operation from a product, the product being a component of the medical system. Once the query is received, the process reads the total PPU data value (v) from the product, stores an investment recovery value (x) to a memory device, clears the investment recovery value (x) from the total PPU data value (v) from the product and provides the user with a credit based on the investment recovery value (x).

Desirably, the cleared investment recovery value (x) coincides with the total value (v), leaving the PPU product with a zero balance (a zero remainder (r)). Any cleared value that is recovered is an investment recovery value (x). The investment recovery data is stored in the memory device and may be added to other data values (x₁₋ₙ) and stored in a user account as part of an integrated or separate database. The investment recovery value (x) that is stored may be held in the system and summed with other cleared values prior to being stored in a remote library, or each cleared value may be sent to the remote library independently. If the cleared value(s) are sent to a remote library, the process includes entering the cleared value(s) into a user account, summing the data (Σx₁₋ₙ=y) in the user account, checking the user account sum against one or more reward criteria and issuing a reward based on the reward criteria. Either the cleared value (x) or the sum of cleared values (Σx₁₋ₙ) may be used to provide credit to the user. The reward or credit may take the form of a cash rebate, product reward or credit toward purchases. The user account may contain additional information about the user.

Transferring the PPU data may be performed through a web based portal and a remote server.

The method described utilizes a product, that is removably attached to a medical system, the product having a component and a read-write (RW) data storage device. Desirably when the RW data storage device is read and the PPU value is zero, the component in the product will no longer operate. Desirably the PPU data that is read, stored and cleared is encrypted.

A product is described having an investment recovery capability. The product has an enclosure having an adaptor for removable engagement with a medical system. There is an electrical circuit within the enclosure, the circuit having at least one port for electrical contact through the enclosure. A component for use in the medical system is suspended within the enclosure; the component requires an activation signal. There is also a RW data storage device incorporated into the electrical circuit, the RW data storage device has a preset number of uses that can be modified when the component is activated.

The RW data storage device having the preset number of uses may be modified by decrementing from or incrementing to the preset number of uses. The modification of the preset number of uses is performed by an electronic controller. Desirably the electronic controller is a computer. The modification of the preset number of uses may be encrypted.

Also described herein is a medical system using the investment recovery method and apparatus. The medical system has a product with an investment recovery capability, the product having a RW data storage device, an activatable component of a medical system and an electric circuit all contained within the enclosure. There is also an electronic controller for operating the product, the electronic controller being able to read and write data to a RW data storage device incorporated into the product and additional adjunct systems as needed for the operation of the product in order for the medical system to perform its intended function.

Apparatus for use with the present invention include items having a pre-set number of uses, with a mechanism for the pre-set uses to be monitored and decremented with each use. The use value of the medical device are the number of uses remaining on the medical device at any given time. The medical device may be one where sterility is required, or where each device is only fit for use for a limited time. If the time expires or it becomes undesirable to continue using the medical device for any reason, the medical device use value may be recovered and credited to the user, thus allowing the user/purchaser to recover some amount of his investment before the medical device is discarded. The recovered investment may be credited back to the user/purchaser, or converted into an award through an award program. An optional database may be used to track a user's accrued use value from one or more medical devices and other information of value to the user, purchaser or supplier.

In the following paragraphs, various aspects and embodiments of the method and apparatus will be described. Specific details will be set forth in order to provide a thorough understanding of the described embodiments of the present invention. However, it will be apparent to those skilled in the art that the described embodiments may be practiced with only some or all of the described aspects, and with or without some of the specific details. In some instances, descriptions of well-known features may be omitted or simplified so as not to obscure the various aspects and embodiments of the present invention.

Parts of the description will be presented using terminology commonly employed by those skilled in the art to convey the substance of their work to others skilled in the art, including terms of operations performed by or components included in the rebate system. As well understood by those skilled in the art, the operations typically involve reading, storing, transferring, crediting, summarizing, refunding and otherwise manipulating data associated with a pay-per-use system. The term system includes general purpose as well as special purpose arrangements of these components that are standalone, adjunct or embedded.

Various operations will be described as multiple discrete steps performed in turn in a manner that is most helpful in understanding the present invention. However, the order of description should not be construed as to imply that these operations are necessarily performed in the order they are presented, or even order dependent.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

The present invention relates to apparatus, systems and methods for tracking the number of uses on a disposable product having a preset limit to its life span, and crediting any unused quantity of life span back to the user. This is particularly valuable in the medical device field where products are sold to users in a “pay-per-use” (PPU) arrangement.

A product described herein refers to a part of a larger medical system, The product contains a data storage device able to record and track pay-per-use (PPU) data. The product is also referred to herein as a medical device, and may be denoted by MD in some drawings.

Changes to the data values as described herein are primarily referenced by their assigned variables. The total PPU data value of the product at any instant it is “looked at” is denoted by (v). Some or all of the PPU data may be removed from the total value (v) at any time and may be credited to a user account value (y) or otherwise may be attributed to a customer. The amount of PPU data removed from the total value (v) in a single removal step is the investment recovery value (x), or obtained value (x). The investment recovery value (x) can be any amount of the total value between zero and the entire value (v). Either the investment recovery value (x), or the user account value (y) is used for the crediting or rewarding programs. When an investment recovery value (x) is removed from the total PPU data value (v) the remainder is (r), which may be any value. Desirably, the remainder value (r) is greater than or equal to zero, however implementation of the present invention allows for the PPU data and variables to be arbitrarily defined in any fashion so long as the data can be reliably processed and recorded.

The medical system includes various adjunct systems as required to operate the medical system for its intended function. However the medical system cannot perform its intended function unless the product (medical device) is properly installed. Adjunct systems are not described in detail herein, as they are well known and understood by those of skill in the art.

In one embodiment, the entire PPU value (v) is obtained (or read) from the medical device, deleted from the medical device as the investment recovery value (x) (thus, in this example, v=x), and credited back to the user. The process of obtaining (reading), deleting and crediting may be done in any order.

Obtaining the value (v) of the PPU component entails accurately determining what remaining uses the PPU medical device has. These unused PPU events represent the unspent portion of the user's investment in the medical device. To continue to use the PPU events or properly credit them back to the user, the value (v) of the PPU device must be obtained.

The storing of the data may be done on any memory device. The memory device may be part of the medical system, a separate computer with data communication to the medical system, or may be maintained on a remote server. The user may attach a portable data storage device (such as a flash memory device) as well.

Crediting the investment recovery value (x) back to the user provides the cost recovery opportunity for the user. Regardless of the reason why the user wishes to recover unused PPU life of the product purchased, the user has an economic interest in having any unused PPU investment returned. The crediting can be done through a variety of methods, such as offering the user a cash refund, coupon for product or services, discount on future purchases or any number of ways that consumers are given rewards, discounts and rebates today through any well known and understood financial and consumer services.

A deleting element may be used so the user does not gain a double credit. The user should not be allowed to claim the benefit of getting a credit for the remaining PPU value, and then use the device to treat patients or perform the intended operation or function of the medical device and in essence get “free” uses from it. Similarly, it would be unreasonable for the user to be able to obtain credit from the PPU events that have been used.

In one embodiment, the use value (v) of the PPU component can be monitored and decremented using a computer. The computer would communicate with a memory device on the PPU product to track the number of uses the product has remaining. For these electronically monitored systems, a computer could obtain the remaining PPU value (or subset of the remaining PPU value), delete it from the data storage device, and credit it to the user (e.g., as an investment recovery value (x)) via a computer program. The operation could be performed in any order, so long as the computer obtains, credits and decrements the appropriate number of uses remaining on the PPU component. In this embodiment, this computer or another computer retains a PPU bank, allowing the user to collect unused PPU from the product. When a user stores enough recovered values (x) on his computer, the user can generate a PPU credit code to send to a supplier for a reward. Alternatively, the product may use a mechanical counter that is machine read by a mechanical reader, or monitored through a verifiable human system.

In referring to the accompanying drawings, it should be understood the drawing figures are provided to enhance the description provided. Elements shown in the figures are not necessarily illustrated to scale with respect to other drawings, or other parts within the same drawing. Nor should the parts or figures be taken in any absolute sense of actual design elements other than as illustrations of embodiments for the purpose of understanding the disclosure herein.

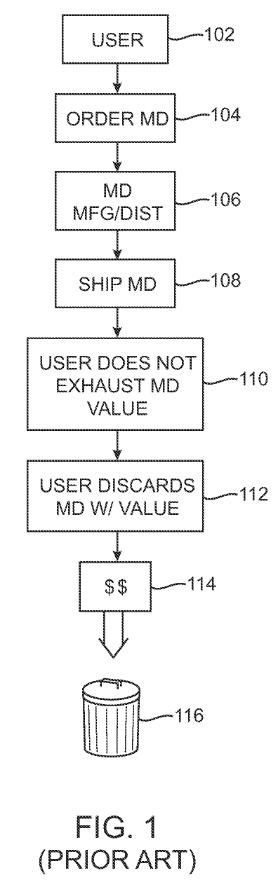

FIG. 1 illustrates the prior art of ordering and using a PPU component. A user or purchaser 102 of a medical device MD can place an order 104 for a medical device through any means acceptable to the medical device supplier. Frequently purchases are made over the internet, by phone or mail, or even in person by having a service representative visit a doctor's office, or having a physician visit a supplier at a place of business, trade show or other forum offering the opportunity for a face-to-face encounter. Once the medical device is ordered, the device manufacturer or distributor secures the ordered device 106 and ships them to the consumer 108. In the process of using the medical device, the user may not exhaust the number of uses paid for before it becomes time to replace, recycle or discard the device 110.

Replacing the medical device prior to the exhaustion of the prepaid uses on it may occur for any number of reasons. Where the medical device is an attachment to a larger medical system, it may become necessary to change the medical device on a daily basis, or with each new patient, it may have to be replaced because of a need to be sterile for each patient, or because the device is restricted to one type of use, or can only be used for a set multiple and not enough multiples remain to complete a whole set. Regardless of reason, the medical device would not be usable again even though it had some previously paid uses on it. Currently the user or purchaser would simply discard the device or return it to the manufacturer for recycling and lose the remaining value 114 on the device. Symbolically, this is akin to tossing the remaining value 114 into the trash 116.

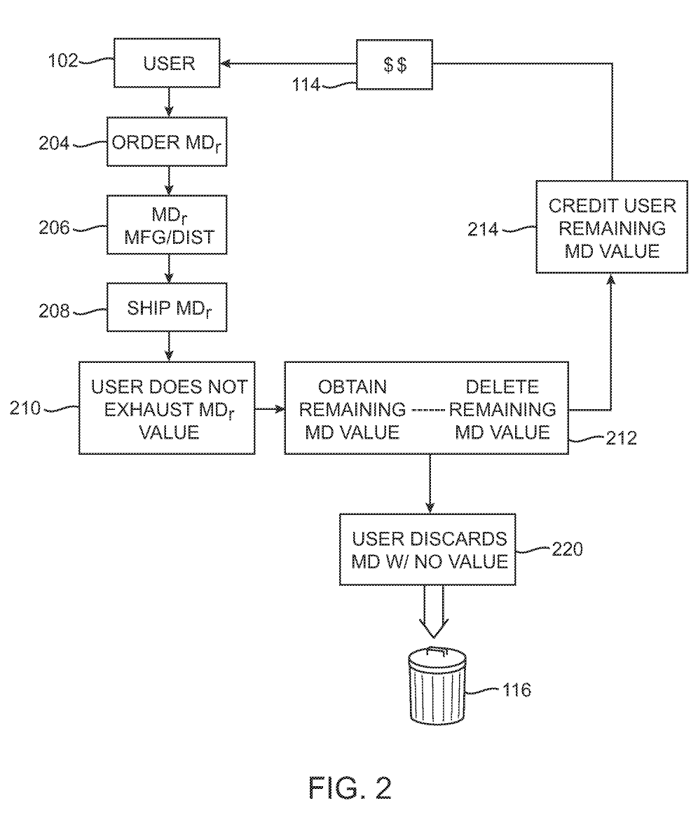

In accordance with an embodiment, if recovery of investment value is incorporated into the product life cycle, the path through which a user or purchaser obtains a new medical device is similar, though the end of the product life cycle is different. Once again the user 102 places an order for a PPU medical device. This time the PPU medical device is one having a recoverable use value MDᵣ (FIG. 2). The manufacturer or distributor provides 208 the MDᵣ to the user. When the user does not exhaust 210 the use value on the medical device MDᵣ, the user may engage in the investment recovery process. When the user decides to change out the medical device MDᵣ, the user can select the recovery process 212 to obtain the part or all of the remaining value (v) on the MDᵣ. Inside the process box 212, the user is able to obtain, delete and gain credit in the form of the investment recovery value (x), for the selected value 214. The user may then discard or recycle 220 the MDᵣ without sacrificing or losing any paid investment in the medical device MDᵣ. The investment is returned 214 to the user in the form of a credit, reward or other vehicle having value to the purchaser or user.

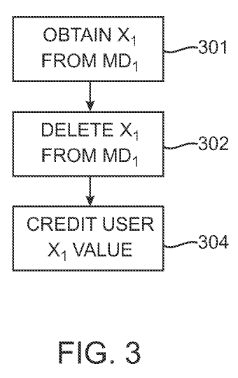

In one embodiment, there is a method for recovering investment in a PPU medical device having a recoverable use value (FIG. 3). The use value of the medical device refers to the remaining PPU value (v) on the device at the time it is processed for investment recovery. The use value may be the same as the original PPU value (v) when the device was new, or any lesser value. The user may select to recover any amount of the use value on the device. The desired recovery value is the obtained or investment recovery value (x). Once the user recovers the investment recovery value (x), that same investment recovery value (x) is cleared 302 from the medical device. The investment recovery value (x) is then credited to the user 304.

The investment recovery value (x) obtained and cleared may be the same, or the investment recovery value cleared may be less than or more than the total use value (v) stored on the product. The order of the obtaining, deleting and crediting of investment recovery values (x) is not particularly important. The investment recovery values (x) recovered are removed from the device so the purchaser or user of the device does not receive an inappropriate double benefit of receiving a credit for the recovered use value and having the advantage of still being able to use that which he or she has been given credit for. Thus the method includes the crediting of the investment recovery values before the investment recovery values are deleted from the medical device, or for the three basic steps to be performed in any order, so long as all three steps are performed.

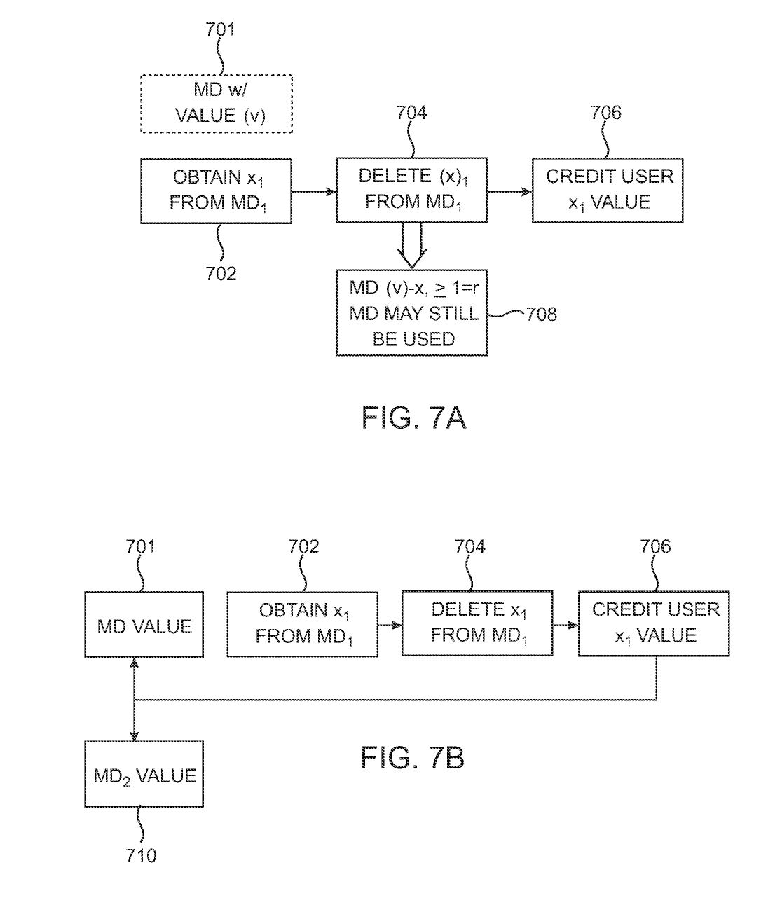

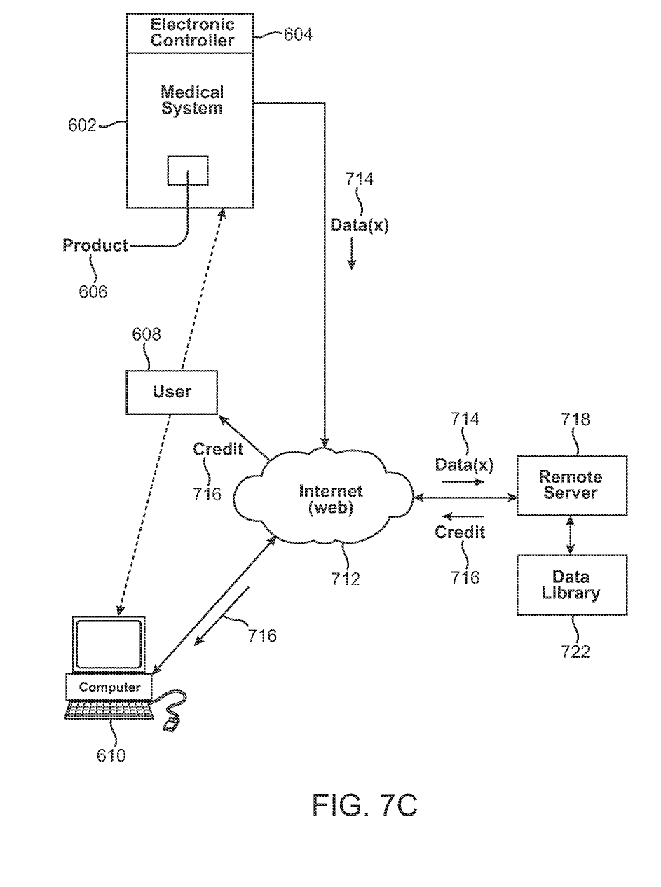

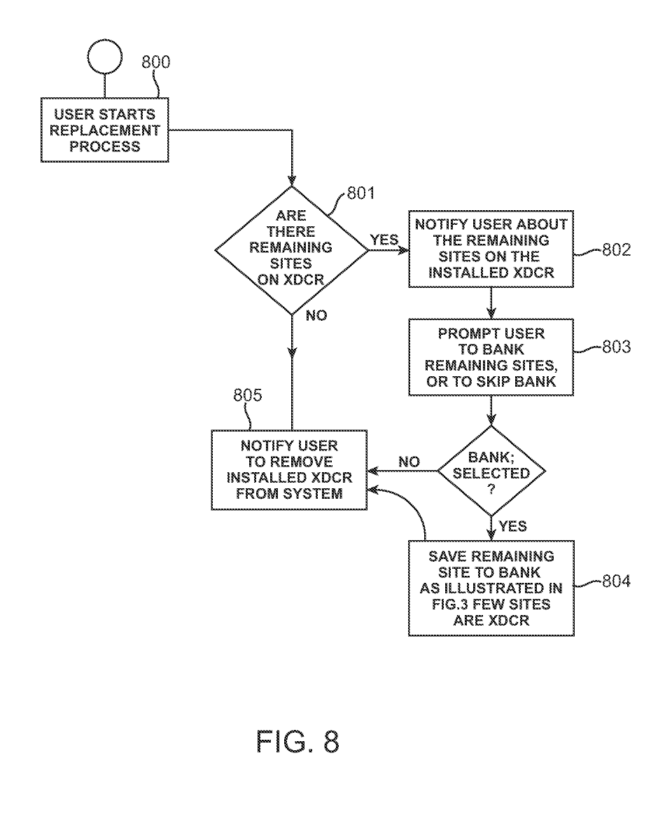

It should be understood that the method provided herein does not require that the value (x) obtained and deleted from the medical device be the same as the total available PPU value (v) remaining on the medical device (FIG. 7A). When a user requests information from the medical system as to value (v) of the PPU device, the computer performs a query and reads the total PPU data value (v) 701. The user can command the computer to execute an investment recovery operation. The computer obtains an investment recovery value (x₁) from the product MD₁ for use with a medical system 702, deletes the investment recovery value (x₁) from the total PPU data value (v) 704 and provides a credit to the user account (y) based on the investment recovery value (x) 706. If the investment recovery value (x) is less than the total PPU data value (v) (v−x>0) then the product MD may still be used.

It is within the scope of the present invention for the use value of the medical device to be decremented by some desired investment recovery value (x), while the actual number of remaining uses (PPU data value (v)) was greater than the investment recovery value (x). Desirably the investment recovery value (x) does not exceed the PPU data value (v), though the investment recovery value (x) may be equal to the PPU data value (v). In this scenario, the medical device has a remainder use value (r). So long as the remainder use value (r) is greater than zero, the PPU medical device may still be used. Once the process is completed, the user may initiate a new command to recover any remaining use value (r). The use value remainder (r) now becomes the total PPU data value (v) available.

The credit 706 obtained for a user from a first product (e.g., in step 702) can be used to increase the total PPU data value (v) of a new, different product 710 (FIG. 7B). Alternatively, the use value (v) of the product MD may be increased through these methods described herein. Thus the credit to a user account (y) can be used to purchase additional uses for either an existing product MD₁ or a new product MD₂.

FIG. 7C shows the operation of crediting the obtained value (x) through an internet portal to a computer system 718 physically removed from the user 608. Note the internet portal may be any remote connection from the user system to the medical system 602 or computer system 610 conducting the crediting of the obtained value (x) to the user 608. The remote system 718 may provide additional capabilities to the crediting of obtained values. The remote system 718 may have a database and/or library 722 that includes detailed user information, and allow the recovery method to anticipate the user's needs based on past use, user designated preferences, or programmed parameters of the system controller. The system 718 can track and monitor the user's account A and issue credits or rewards 716 on demand from the user, or as part of an automated system of dispensing rewards.

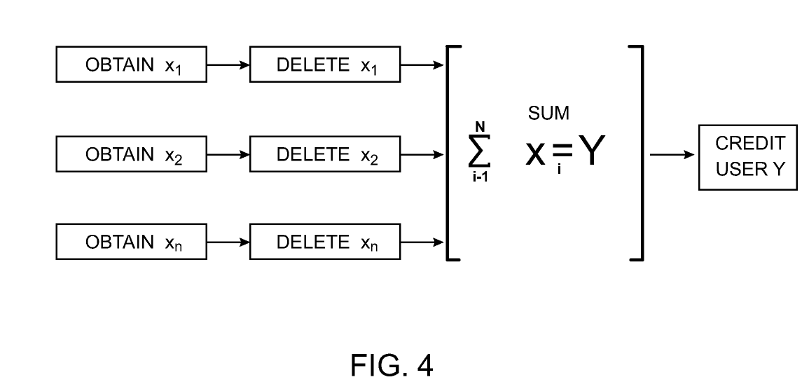

In another embodiment, a user may accrue more than one investment recovery value (x) before receiving credit for the sum of the investment recovery values. This is illustrated in FIG. 4. The user may obtain a first investment recovery value (x)₁, delete the investment recovery value (x)₁ from a first PPU medical device MDᵣ₁ and store that investment recovery value (x)₁. Then the user may recover a second investment recovery value (x)₂ from a second PPU medical device MDᵣ₂, delete the second investment recovery value (x)₂ from the second PPU medical device MDᵣ₂, and add the second investment recovery value (x)₂ to the first investment recovery value (x)₁. The user may perform this operation as many times as desired, represented as obtain (x)ᵢ, delete (x)ᵢ and sum (x)₁₋ᵢ. The sum of the investment recovery values (x) becomes the value of the credit to the user account (y). The steps of obtaining one or more investment recovery values (x), along with deleting the investment recovery values and crediting them may be done by the user, or the manufacturer, seller or other party having authorization to do so. A medical system having an apparatus, system or method as described herein may automatically process the investment recovery process without a user request when a particular parameter is detected.

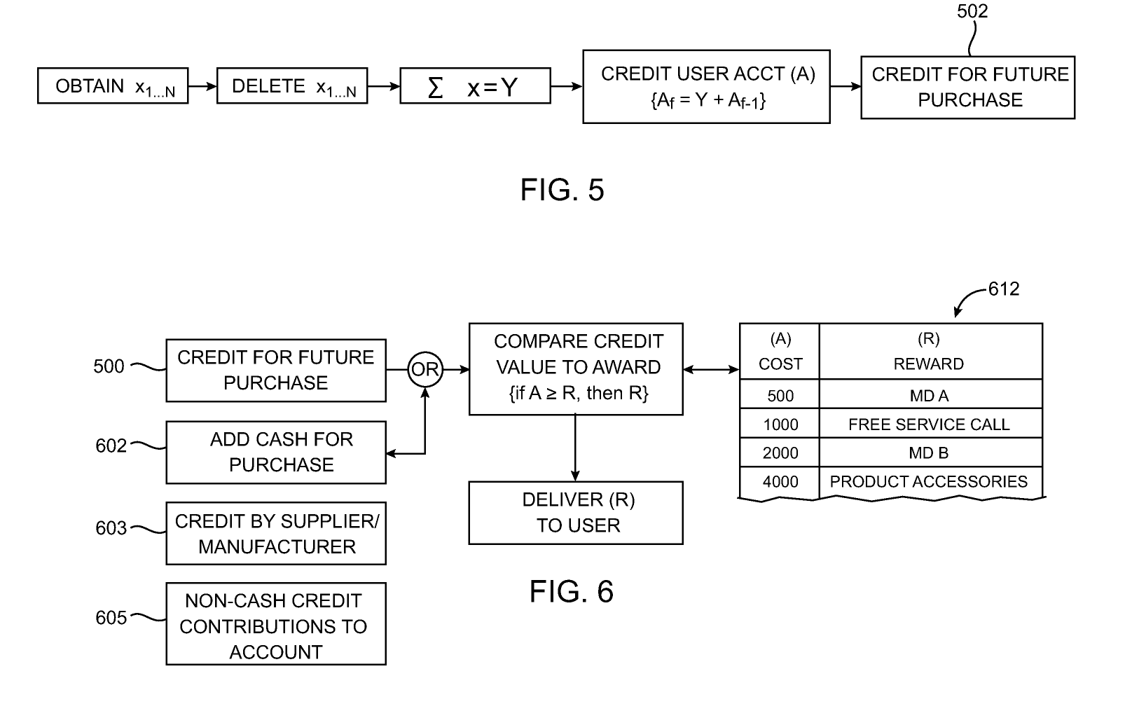

The methods described herein may also incorporate the use of a database, ledger or other bookkeeping method for storing credits for a user. In FIG. 5, the method of obtaining and deleting the investment recovery values (x) from medical device may follow the path or description of any route described herein. The crediting of the investment recovery values (x) is handled through a bookkeeping device A like a database or ledger. A user account corresponding to the identity of the user who is accruing investment recovery values (x) may be saved in the database A. When new investment recovery values (x) are added to the database the investment recovery account is updated so the user account value (y) has current information on the number of use values to be credited back to the user.

The user account value (y) may be increased or decreased as shown in FIG. 6. The user account 500 may have value added to it through other means such as cash for purchases 602, credits from supplier or manufacturer 603, or other non-cash credit contributions to the user account 605. The user account A may be used to purchase additional medical devices, or other supplies and services such as may be available to the user account. The user may request to have credits converted into product/services, or the system may provide an automatic dispensing of products/services when the user account reaches a predetermined value. The amount of the user account A may be compared to the value of product/services in a table 612, then any desired corresponding reward R from the table 612 may be selected and provided to the user 614. Alternatively the user may add a cash value 606 to the user account to purchase additional products/services. In the simplest format, the user may simply “cash out” the user account by converting the use value of the user account A into a cash equivalent. Desirably, there is another lookup table or parameter to guide the conversion of the user account value into cash or other non cash rewards.

The stored information on the database may include any amount of other information besides the number of unused uses of the disposable products. A user may store and redeem the unused uses of products in the database for additional products, services or other awards offered by the product sponsor or manufacturer.

The user command to recover PPU value may be integrated into other interactions with the system as shown in FIG. 8. One alternative interaction that may trigger the recovery of the PPU value may be the act of replacing the product 800. The system detects that there is value remaining on the product 801. The system could automatically recover the PPU value, or as shown in FIG. 8 the system could query the user to determine if recovery of the PPU value (recovery of PPU value, banking of sites or site banking are three terms which describe the same operation) is desired at this point 802 & 803. Having determined that recovery is desirable the system recovers the value 804. The system may then proceed with the requested replacement of the medical device 805.

When the product is used, the use counter in the electronic chip is decremented appropriately. Anytime the user desires, the remaining number of uses (the unused life total) or the remaining value of the product may be transferred to a computer having a database for storing the user's account information. The database stores the accumulated number of uses the user has transferred from the products. The user may redeem the number of uses in the database for additional products, services or awards as may be available from the manufacturer, sponsor or authorized agent of the disposable product, database or other party.

The product as described herein desirably attaches to a non-disposable product or a system platform. The system platform may be a medical device for therapeutic medical procedures, or other product wherein tracking and crediting uses of disposables is desirable. In one embodiment, the medical system has an electronic controller, such as a computer, for controlling operations of the system and decrementing the value of uses in the product. The product may be a “dumb” product or it may possess its own electronic intelligence (some kind of processor capability). In both embodiments, the product possesses a data storage device, like an electronic chip, with a preset use limit programmed into it. The uses may be decremented each time the product is used. The decrement process is controlled through the medical system.

In another embodiment the PPU product is a non-disposable product. The decrementing of uses is a means for tracking the use of the product and a way to charge per use. The user may have value added to a non-disposable PPU device in the same manner as doing a credit exchange. In this embodiment, the credit of the user account would correlate to additional uses to the PPU product. Desirably the remote server can provide increases in credit value to the PPU product remotely at the same time a user is making the request for credit to reward exchange. The credit to reward may also be done with a service call or by having the PPU product sent to the manufacturer or authorized agent for PPU value increase.

Alternatively the product may contain some level of electronic intelligence and may self-decrement with each used. In this embodiment it is also possible for the product to operate independently of the medical system (the product may be a battery operated device with its own on/off switch).

Transferring the unused portion of the product life to a computer database may proceed through any variety of communication protocols. The user may initiate the process through which the electronic counter on the product is read, and then the unused remainder is transferred to a computer, or other memory device (persistent/volatile). The transfer may be done electronically, from the disposable directly to the database, or through one or more intermediate electronic devices. Desirably the user can send a query to “bank” the remaining PPU value of the product from the UI of the medical system, then the medical system will contact with the remote server and carry out the download of unused site information automatically. For example the transfer may occur from the disposable product to a system platform, then from the system platform to one or more internet hubs and then to a remote computer. In another embodiment, the transfer of use information may flow from the disposable product to a host system, then converted into a user ASCII string, which the user then loads into the database. The user may load the information into the database by calling the information in, or logging into a website through any accessible web portal. The user may send the ASCII string in through the mail or any other convenient means of delivery to a remote site.

Once the previously unused life of any disposable product is returned to the remote computer or a host system, the user's identification is confirmed and the data is stored in a user account reflecting that users purchases of disposable products and uses. The unused life total of any transferred product is converted into new products or services at the discretion of the user or the manufacturer of the disposable product. The new product or service is then supplied to the user on request, or through an automatic processing queue.

Alternatively, the database may be stored on the same system as the host system. The unused life of the disposable product may be uploaded to the host system and stored there. The user may redeem the unused life total in the database by contacting the seller or distributor for the disposable products and redeem the values in his own database.

Desirably the database values and the upload and download of value information are secure from user tampering, or tampering by third parties. Data may be encrypted to preserve data integrity and prevent users or third parties from abusing or “ripping off” distributors and suppliers. Similarly, the electronic chip used in the disposable product is also protected against unauthorized uses, decrease or increase of its use value.

In one alternative embodiment, the crediting of the remaining PPU value can be maintained in a database under the control of the manufacturer or seller of the PPU product. In this embodiment, the database may be used to track a wide variety of information on the user, and/or the user's use of the PPU devices. Data may include payment information and authorization (such as an open purchase account), user preferences for PPU products, type of business, or any other information the provider of the PPU medical device may choose to keep track of.

In addition to user information, the database can be used to track the user's supply of PPU medical devices (stock on user site, available stock to send to user, predicted time needed to replenish user stock, etc. . . . ). The database may be connected to the crediting method to ensure the user is well supplied with the desired PPU medical devices, and that all credits, coupons or other rewards are applied to minimize the cost burden to the user. It would be desirable for the user to be able to view his or her account balance on line (or by written statement/phone call) such as any consumer may view financial or other confidential information through a bank account, credit card service or the like. The user can request a current balance and have that balance displayed (or otherwise have that information relayed to him or her). Desirably the user would have to authenticate his or her identity to the database system to protect the user against fraud, identity theft or other unscrupulous conduct. The user may then conduct transactions through the web portal (or other contact vehicle) to obtain credits, refunds or rewards for the PPU values accumulated and stored within the customer database.

The user may then request a new PPU medical device, consumable or other reward offered through the customer database. The user's account is decremented the appropriate number of PPU values (which can be points, credits, or other numerical representations of his or her accumulated PPU value level). The requested reward is then shipped to the user. Desirably the seller's PPU value system is connected and operates in conjunction with an inventory control program to ensure adequate levels of supply and production of the ordered and reward type PPU products.

In another embodiment, the user may be able to obtain the remaining PPU value through the system incorporating the PPU medical device. The system may display the remaining PPU value on a screen or other GUI device for the user, or produce the view on command from the user. Desirably the PPU device has an encrypted counter to prevent tampering. The user may desire to decrement the PPU device any amount up to the remaining total and have that converted into a credit for later use.

For instance, the PPU medical device may start with 500 uses on it. The user may operate the medical device through 450 uses, and then select to credit 48 of the remaining 50 uses. In this manner, the user may retain the medical device for two more uses, such as to demo the product for a potential client without having to use a new product for customer development efforts. The user may of course convert the entire 50 uses to credit. Electronically the system decrements the memory device on the PPU medical device to reflect the users crediting value. So long as the remaining PPU value on the medical device is greater than zero (0), the PPU medical device may still be usable. If the PPU medical device has a remaining use value of zero (0), then the device is no longer functional and may be discarded, recycled or recharged.

An illustration of a medical system using the various embodiments of a PPU medical device is now described. For illustrative purposes only, the system 900 is shown with a main compartment 904, a suspension device 902 and a therapy head 910. A single graphical user interface (GUI) 906 is located near the therapy head 910. The system is desirably mobile and fitted with castors 912. The therapy head 910 includes an energy applicator such as a transducer 1038. The suspension device 902 is typically a mechanical arm. The third component is a therapy controller in electronic communication with the therapy head and the suspension device supporting the therapy head. The therapy controller is adapted to monitor the position and/or energy delivery of the scan head while providing guidance for positioning the scan head.

Within the system main compartment 904 are a variety of sub systems used to maintain and operate the device. These subsystems (not shown) are arranged to provide data communication between the therapy head 910 and the main compartment 904, as well as power, coolant, and system monitoring feedback for safety.

### Therapy Head

The therapy head (also called the scan head) is a housing containing an energy applicator, and any additional devices needed for the effective operation of the energy applicator during a therapeutic procedure. Multiple designs may be selected from for use in the scan head sub system. The therapy head is generally configured as an inverted cup or bell, having a chamber with an aperture at the bottom of the therapy head. The chamber may be divided into two sections, forming an upper chamber and a lower chamber, with a seal between them. The upper chamber contains such electronic and motor drive units such as are needed for the manipulation and control of the energy applicator. The lower chamber contains the energy applicator, ultrasound coupling fluid and such sensors as are deemed necessary for the proper operation of the system.

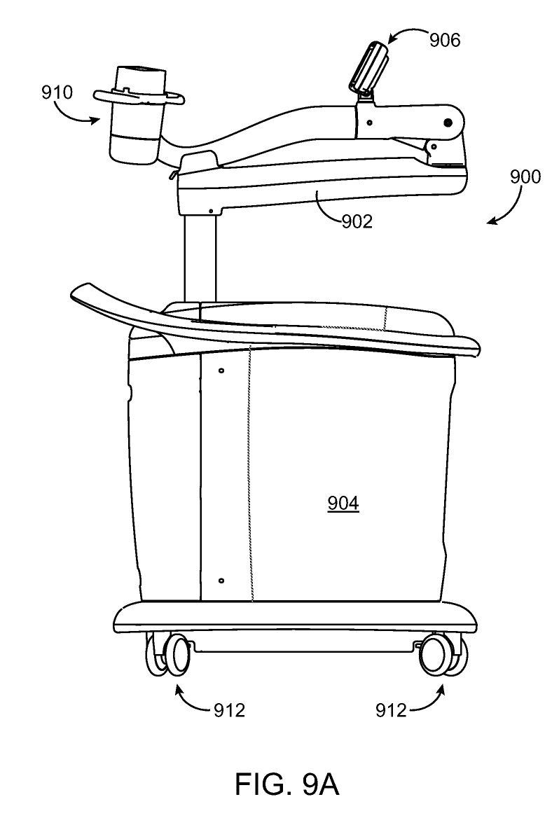

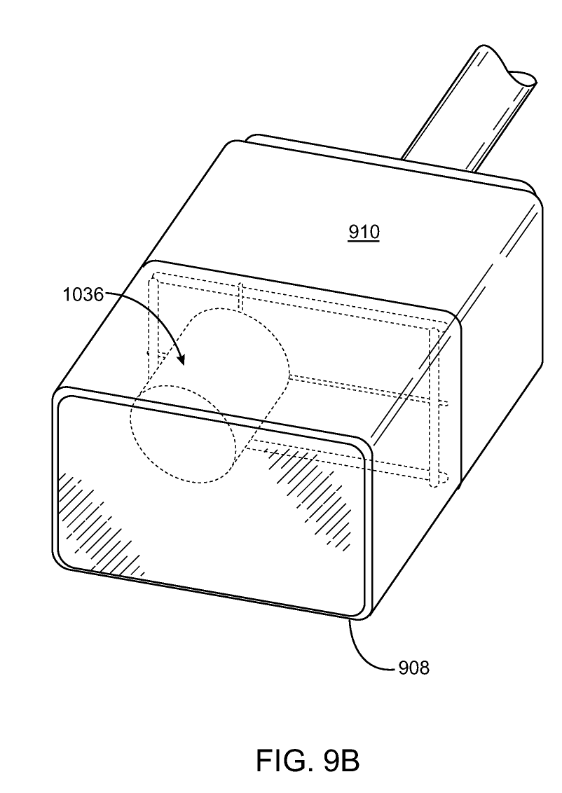

Preferably the exterior design of the therapy head is ergonomic so a user may hold the therapy head in one hand while moving it, or orientating it against the skin surface of a patient. The ergonomic fit is for holding and guiding, but not for carrying the weight since the therapy head is intended to be supported by mechanical means. The therapy head 910 (FIG. 9A) is connected to the mechanical arm 902. From the top end of the therapy head there are a plurality of connection lines (not shown) used to connect the components inside the therapy head with the corresponding electrical subsystems and cooling system in the main compartment. The connection lines may be integrated into the mechanical arm 902, or run independently of the suspension device.

The partition between the upper and lower sections of the therapy head is desirably fluid tight, but having one or more apertures to allow electrical or cooling fluid access into the lower chamber. The apertures may also be used for mechanical linkages between a motor assembly in the upper chamber, and the energy emitter in the lower chamber. Linkage apertures may be omitted if the energy applicator (transducer module) may be moved without direct mechanical linkage, such as with a magnetic connection using magnets on each side of the partition to slide the transducer module over the partition.

### EXAMPLE I

### Consumable Transducer Product

An example of a computer implemented method for investment recovery in a medical device is now provided. In this example, the component of the PPU product is an ultrasound transducer, for use with a high intensity focused ultrasound (HIFU) medical system. A product is provided to a user having a preset PPU value. The transducer is the component of the product, and a RW data storage device is incorporated into the product. The RW data storage device holds the preset PPU value. The product is adapted to mechanically engage with the HIFU system, which does not have a HIFU transducer when the product is not engaged. Thus the system requires the product to operate regardless of whether or not a PPU investment system is used.

The recovery can occur any time a user requests a completion or banking of the PPU device consumable. The response is the account balance maintained on the system increases, while the status of consumable is updated to show it is partially or entirely consumed.

The software provides control on system UI that allows user to initiate transfer, responds to control activation, encrypt status showing consumable is partially or fully consumed. The software then writes the status to consumable. Desirably the data operations to the consumable are encrypted. An update to the non-volatile system storage is done to include additional value stored on the medical system. Then transfer the unused value from the medical system to the user.

This will change the banked value on the system to a lower value or a depleted value (v=0) and a code representing equivalent value may be supplied to the user. This code will remain viewable on the system until the next time the user requests a code.

The user may request a transfer of the banked value from the system at any time. The account balance maintained on the system decrements, and a code is supplied to the user.

The software provides control on system UI that allows user to initiate transfer. There is a response to control activation and then a code is generated representing value stored on system, serial number of code, and system ID. Desirably the code is encrypted. The code is displayed to the user. An update of the non-volatile system memory is done to remove value stored on system.

A transfer of the unused value from the user to a supplier (or other designated recipient) is done. This allows the user to update the banked value at the supplier using the code generated by the system. The code can be transferred via a web based portal, e-mail, or other means (even physical media like post).

The system previously provided a code to the user. The user may provide the code to the supplier, or desirably the system automatically provides the code to the supplier. Desirably the operation is executed through either a web based portal and remote server. Alternatively the user may send the code to the supplier through any acceptable method.

Once the code is received by the supplier, through either the web based portal and server, or through other means, the user's banked value at supplier is increased.

The software displays a web page to allow a user to login with their username and password. The remote server validates the username and password and displays web page(s) allowing the entry of the code. The supplier side server decrypts the entered code and validates that it is an unused code from a system owned by that user. Note that the serial number and system ID will be used to track which codes have been received from each system. If it were used in conjunction with a transfer of unused value from the PPU product the validation would be that it was an unbanked consumable. A data library containing user account information is updated to increase the balance for next time user views it.

Alternatively, the user can request transfer of banked value and the account balance maintained on the system will decrement by some value, while the banked account value at the supplier increases by the same amount. The software provides control on system user interface (UI) that allows user to initiate the transfer. There is a response to the control activation producing a code representing value stored on system, serial number of code, and system ID. Desirably the code is encrypted. The code is transferred to a supplier. The non-volatile system storage is updated to remove value stored on the system.

On the supplier side, the transferred code is decrypted if necessary and validated. The user account database is updated to increase the balance for the next time the user views it. A transfer of unused value from the PPU product to the user occurs.

This will change the PPU product (consumable) to be partially or completely depleted and a code representing an equivalent value will be supplied to the user. This code will remain viewable on the system until the next time the user initiates a similar transfer.

The user may request completion or banking of the PPU product or consumable. The status of the consumable is updated to show it is either partially or entirely consumed, and a code is supplied to user.

The software provides control on the system UI that allows user to initiate transfer and responds to control activation. A status is provided showing what portion of the consumable is consumed. Alternatively it may show what portion of the consumable remains. The status is written to the consumable. The system generates a code representing value stored on consumable, and serial number of consumable and the code is displayed to the user. Desirably the code is encrypted. The unused value derived from the consumable is provided to the supplier and decrypted if necessary. This will change the consumable to be partially or completely depleted and update the banked value at the supplier from a system electronically connected to the supplier. The connection from the system to the supplier can be a web based portal to a remote server or other data exchange connection.

Alternatively the user may request completion or banking of the consumable. The status of the consumable is updated to show it is partially or entirely consumed. The account value in the remote library is increased accordingly.

The system provides control on the system UI that allows user to initiate the transfer. The system responds to a control activation, and a status showing the consumable is partially or fully consumed. The status information is desirably encrypted. The status is written to the consumable, and a code is generated representing the value stored on the consumable, and the serial number of the consumable. The code is transferred to the remote library of user accounts and decrypted if needed. The code is validated. The library database is updated to increase the balance for the next time a user views it. The PPU product consumable is then shipped to the supplier where the supplier will read the value off the consumable for storage in the library.

In another alternative embodiment, the user can remove the unspent consumable and return it to the supplier. The supplier then increases the banked value of the remote server.

The supplier can insert the consumable into a reading device that can determine the remaining PPU value of the consumable and provide the information to the library. The library is once again updated so the user can view his balance.

Finally, the user can request her current balance from the medical system or through a web based portal. Here there is a control on the UI that allows a user to request balance information. The balance is retrieved from non-volatile storage on the system or from the remote library. The balance is displayed and the user is given the option to convert some or all of her balance into a credit for a product reward, cash or other device of value to the user.

### EXAMPLE II

### Use CAP

The device uses one “cap” per patient. The cap is the cover at the bottom of the treatment head and is the part that comes into contact with the patient. Each cap contains electronics that tracks how many sites have been treated. There are several versions of the cap—each with a different number of sites. For example, a customer could buy a 50 site cap for single areas, or a 100 site cap to treat a larger area or multiple areas. The customers would pay a certain amount for each cap, paying more for caps with more capacity. There are similar revenue models for products on the market today, but they all have a common problem: if the customer treats fewer sites than the capacity, they “waste” the remaining capacity on the disposable part. To solve this problem, the system provides a memory bank of unused sites and would keep track of the number. For example, if a 50 site cap is used to treat a patient whose treatment area is covered by only 40 sites, the remaining 10 sites would be stored in the system memory bank. The customer could have several methods for retrieving sites stored in the memory bank:

A. Service call: A service rep could visit the customer, record the number of sites in the memory bank, clear the memory bank, and give the customer new caps with a number of sites equivalent to what had been stored in the memory bank, plus any additional the customer may purchase for value.

B. Retrieval Cap: The customer could buy a retrieval cap at a low cost and retrieve sites from the memory bank without a service visit. For example, the customer had over 50 sites in the memory bank, they could buy a 50 site retrieval cap for a nominal fee and install it. The retrieval cap would erase 50 sites from the memory bank and allow the customer to treat 50 sites with that cap.

C. Rolling site bank: The system could come with some number of sites already loaded in the memory bank. If the customer treats a patient with fewer sites than are on the cap, the excess sites are stored in the memory bank. If the customer needs some extra sites at a different time, they can draw from the excess in the bank. For example, if a system had 10 sites in the bank and a customer had a 50 site cap and 55 sites to treat, they could treat all 55 sites and draw the bank down to 5 sites from 10.

### EXAMPLE III

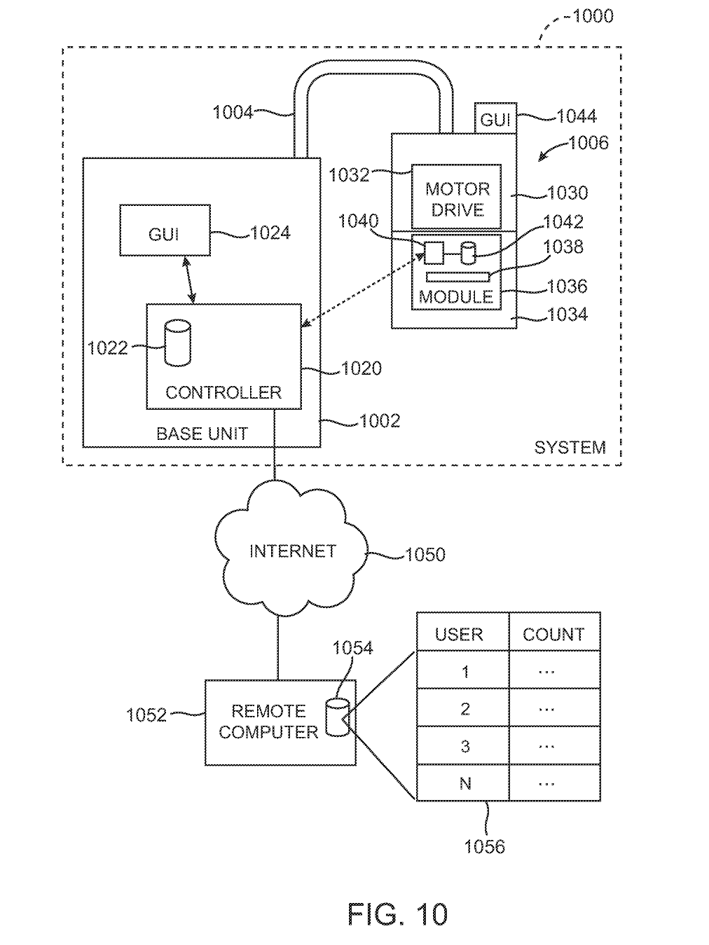

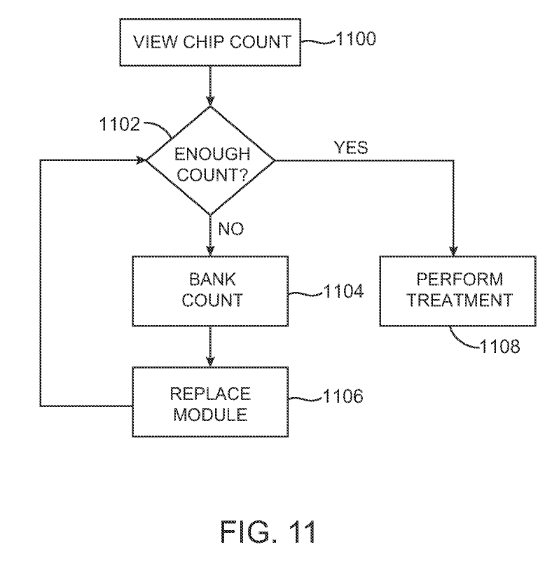

FIG. 10 shows a specific embodiment of a medical system 1000 in accordance with an embodiment. In the embodiment shown in the drawings, the medical system 1000 is a high-intensity frequency ultrasound (HIFU) treatment device, as an example, such as is described in U.S. Published Application No. 2005/0154431, filed Jul. 14, 2005, and entitled “Systems and Methods for the Destruction of Adipose Tissue.”

The medical system 1000 includes a base unit 1002 connected by a mechanical arm 1004 to a therapy head 1006. The base unit 1002 may include a number of different features, including a water chiller and/or degasser and/or other operational components that may be utilized with the therapy head 1006. In the embodiment shown in the drawings, the base unit 1002 includes a controller 1020 having, or otherwise associated with, a data store 1022. A graphical user interface 1024 is provided for viewing or controlling operation of the controller 1020.

The controller 1020 may be a standard control (i.e., a device or mechanism used to regulate or guide the operation of a machine, apparatus, or system), a microcomputer, or any other device that can execute computer-executable instructions, such as program modules. Generally, program modules include routines, programs, objects, components, data structures and the like that perform particular tasks or implement particular abstract data types. A programmer of ordinary skill in the art can program or configure the controller 1020 to perform the functions described herein.

The therapy head 1006 includes a dry side 1030 having a motor drive 1032 therein, and a wet side 1034. As described above, the wet side 1034 may include a transducer module 1036, which in this document is the “product” as described throughout this document. The transducer module 1036 includes a transducer 1038, which is the “component” of the “product” as described herein.

In the embodiment shown in the drawings, the transducer module 1036 includes a PPU tracking component 1040 associated with a data store 1042. The PPU tracking component 1040 may be a standard control or any other device that can perform the functions described herein. A programmer of ordinary skill in the art can program or configure the PPU tracking component 1040. A graphical user interface 1044 is connected to the PPU tracking component 1040, either wirelessly or in a wired manner, and controls and/or views operation of the PPU tracking component 1040.

In the embodiment shown in the drawings, the controller 1020 for the base unit 1002 is connected via a network, such as the Internet 1050, to a remote computer 1052. The remote computer 1052 includes or is otherwise associated with a data store 1054. In the embodiment shown in the drawings, the data store 1054 stores information in the form of a table 1056.

FIG. 11 shows a method of utilizing the medical system 1000 of FIG. 10 in accordance with an embodiment. In practice, a user, such as a surgeon or a technician, purchases the transducer module 1036 with a particular number of uses in the module, such as 450 uses. In accordance with some embodiments, such as is described in U.S. Published Application No. 2007/0055156, filed Mar. 8, 2007, and entitled “Apparatus and Methods for the Destruction of Adipose Tissue,” a treatment on a single patient may involve multiple uses of the transducer 1038, such as 50 uses. In treatment, it is desired that the number of uses needed for the treatment be on the transducer module 1036 at the beginning of the treatment, so that the module does not have to be changed in the middle of treatment.

Thus, in accordance with an embodiment, at step 1100, the user views the chip count, or the number of available uses of the transducer 1038 on the transducer module 1036. These uses are stored on the data store 1042, which may be the read-write (RW) data storage device described above. The PPU tracking component 1040 access the data store 1042 to provide this information to the graphical user interface 1044. The PPU tracking component 1040 also decrements the count maintained in the data store 1042 as the transducer 1038 is used.

Thus, after the user has viewed the chip count in step 1100, at step 1102 a determination is made whether or not there is enough counts on the chip to perform a particular treatment. As described above, this count may be, for example, 50 uses. If there is not enough count on the chip, then step 1102 branches to step 1104, where the user may select to bank the count. That is, the user may elect to use the investment recovery procedure described above, and remove the investment recovery value (x) from the data store 1042. In an embodiment, the investment recovery value (x) is moved to the data store 1022 in the base unit. This operation may be performed wirelessly or through a wire, for example, that extends through the mechanical arm 1004.

Alternatively, if at step 1102 a user found there was enough of a count to perform a treatment, the step branches to step 1108, where the treatment is performed.

The counts that are stored on the data store 1022 on the base unit 1002 may be viewed by the user via the graphical user interface 1024. In an embodiment, the counts are moved from the data store 1022 on the base unit 1002 to the data store 1054 on the remote computer 1052. This movement may be done in a number of ways, such as automatically according to a time schedule, in response to the user via the graphical user interface 1024, in response to an administrator at the remote computer 1052, or via some other method.

The remote computer 1052 stores the counts in the data store 1054. In the embodiment shown in the drawings, these counts are stored in a table format, with multiple users and a count kept for each of the users. A user may request a reward, and the remote computer 1052 may access the data table 1056 to determine if the user has appropriate count to receive the reward.

Alternatives may be employed. For example, counts may be sent directly from the data store 1042 on the transducer module 1036 to the data store 1054 on the remote computer 1052. In addition, in embodiments, the graphical user interface 1044 and/or the graphical user interface 1024 may omitted, and counts may be moved automatically or by other methods. For example, the transducer module 1036 may automatically bank counts as a result of a time delay and the number of counts on the device being below a number. So, for example, when the transducer module has not been used for a couple of hours, indicating a treatment is done, then the counts may be automatically banked, or alternatively, a request may be provided to a user for banking of counts. Similarly, counts may be sent from the data store 1022 on the base unit 1002 upon reaching a threshold, or when connected to the Internet 1050, based upon temporal requirements, or some combination of these.

Additional alternative embodiments of the present invention will be readily apparent to those skilled in the art upon review of the present disclosure. The lack of description or the embodiments described herein should not be considered as the sole or only method and apparatus of providing for an interchangeable transducer. The scope of the present invention should not be taken as limited by the present disclosure except as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A computer implemented pay-per-use (PPU) investment recovery method for use with medical systems, the method comprising:
in a medical system, receiving a command requesting a PPU data storage and clearing operation from a product, the product being a component of the medical system;
reading the total PPU data value (v) from the product, the total PPU data value representing a number of permitted uses for the product and being decremented as a result of use of the product;
clearing an investment recovery value (x) from the total PPU data value (v) of the product, the investment recovery value (x) being a subset of the total PPU data value (v); and
storing the investment recovery value (x) to a memory device.

2. The method of claim 1, further comprising providing a user with a credit based on the investment recovery value (x).

3. The method of claim 1, wherein storing comprises:
transferring the investment recovery value (x) to a remote system, the remote system maintaining a plurality of identifiable user accounts.

4. The method of claim 3, wherein transferring the investment recovery value (x) to a remote system comprises:
entering the investment recovery value (x) into a user account;
summing the investment recovery values of the user account to obtain a sum data value (y) for the user account (Σx₁₋ₙ=y);
comparing the sum data value (y) against a reward criteria; and
if the sum data value meets or exceeds the reward criteria, issuing a reward option based on the reward criteria.

5. The method of claim 3, wherein transferring the investment recovery value (x) to a remote system is performed through a web based portal and a remote server.

6. The method of claim 3, wherein the user accounts further comprise information about the user.

7. The method of claim 1, wherein the product is a removable component of the medical system, the component having a read-write (RW) data storage device for maintaining the total (PPU) data value for the product.

8. The method of claim 1, wherein the product will no longer operate when said total PPU data value (v) for the product is zero or less.

9. The method of claim 1 further comprising encrypting the data.

10. The method of claim 1, wherein the investment recovery value (x) read, stored and deleted from the product is less than a total PPU data value (v) available on the product.

11. The method of claim 1, wherein the investment recovery value is equal to the total PPU data value on the product (v−x=0).

12. The method of claim 1, wherein the product contains an ultrasound transducer.

13. The method of claim 1, wherein the medical device is a non-invasive therapeutic ultrasound system.

14. The medical device for use in the method of claim 1.

15. A computer implemented pay-per-use (PPU) investment recovery method for use with medical systems, the method comprising:
in a medical system, receiving a command requesting a PPU data storage and clearing operation from a product, the product being a component of the medical system;
reading the total PPU data value (v) from the product, the total PPU data value representing a number of permitted uses for the product and being decremented as a result of use of the product;
clearing an investment recovery value (x) from the total PPU data value (v) of the product, the investment recovery value (x) being a subset of the total PPU data value (v); and
storing the investment recovery value (x) to a memory device.

16. The method of claim 14, further comprising providing a user with a credit based on the investment recovery value (x).

17. The method of claim 14, wherein storing comprises:
transferring the investment recovery value (x) to a remote system, the remote system maintaining a plurality of identifiable user accounts.

18. The method of claim 16, wherein transferring the investment recovery value (x) to a remote system comprises:
entering the investment recovery value (x) into a user account;
summing the investment recovery values of the user account to obtain a sum data value (y) for the user account (Σx₁₋ₙ=y);
comparing the sum data value (y) against a reward criteria; and
if the sum data value meets or exceeds the reward criteria, issuing a reward option based on the reward criteria.

19. The method of claim 16, wherein transferring the investment recovery value (x) to a remote system is performed through a web based portal and a remote server.

20. The method of claim 16, wherein the user accounts further comprise information about the user.

21. The method of claim 14, wherein the product is a removable component of the medical system, the component having a read-write (RW) data storage device for maintaining the total (PPU) data value for the product.

22. The method of claim 14, wherein the product will no longer operate when said total PPU data value (v) for the product is zero or less.

23. The method of claim 14 further comprising encrypting the data.

24. The method of claim 14, wherein the investment recovery value (x) read, stored and deleted from the product is less than a total PPU data value (v) available on the product.

25. The method of claim 14, wherein the investment recovery value is equal to the total PPU data value on the product (v−x=0).

26. The method of claim 14, wherein the product contains an ultrasound transducer.

27. The method of claim 14, wherein the medical device is a non-invasive therapeutic ultrasound system.

28. A medical system comprising:
product having an investment recovery capability, the product having a RW data storage device for maintaining a total pay per use (PPU) data value for the product, the total PPU data value representing a number of permitted uses for the product and being decremented as a result of use of the product, and an activatable component of the medical system;
an electronic controller for operating the product, the electronic controller being able to read and write data to the RW data storage device, the controller comprising a module for accessing the RW data storage device, clearing an investment recovery value (x) from the total PPU data value (v) of the product, and causing the investment recovery value (x) to be stored to a memory device; and
a plurality of adjunct systems needed for the operation of the product in order for the medical system to perform its intended function.

29. The medical system of claim 28, wherein the electronic controller is a computer.
